(19) United States
(12) Patent Application Publication
(10) Pub. No.: US 2010/0297532 A1
(43) Pub. Date: Nov. 25, 2010

(54) PROCESS FOR WORKING UP IONIC LIQUIDS

(75) Inventors: York Alexander Beste, Ludwigshafen (DE); Veit Stegmann, Mannheim (DE); Matthias Maase, Mendham, NJ (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/811,100

(22) PCT Filed: Jan. 8, 2009
(86) PCT No.: PCT/EP09/50152

§ 371 Date: Jun. 29, 2010

(30) Foreign Application Priority Data
Jan. 9, 2008 (EP) 08150131.4

(51) Int. Cl. C09K 3/00; B01J 32/00; C10M 135/36; C10M 133/04; C10M 133/40; C10M 133/44; C10M 133/46; C10M 133/48; H01G 9/02; H01M 8/08; H01M 6/04; C09K 5/00; G01N 33/00; H01B 1/12

(52) U.S. Cl. 429/498; 252/182.12; 252/364; 502/439; 508/243; 508/244; 508/255; 508/271; 508/270; 508/272; 508/283; 252/62.2; 429/188; 252/77; 252/408.1; 252/500

(57) ABSTRACT

The present invention relates to a process for the work-up of acidic or basic ionic liquids (IL) of the general formula (I),

[A]⁺(1/n)*[Y]⁻ (I)

where [A]⁺ is a quaternary ammonium cation and (1/n)*[Y]n⁻ is one anion equivalent of an n-fold charged anion, which comprises adding a conjugate acid-base pair selected from among compounds of the formula (II),

[A]⁺[X]⁻ (II)

where [A]⁺ has the meaning given for the ionic liquid and [X]⁻ is a conjugate base to the ionic liquid (IL).

The present invention relates to a process for the work-up of acidic or basic ionic liquids (IL) as are obtained, for example, from industrial processes in which a suitable conjugate acid-base pair is added to the ionic liquid (IL).

Ionic liquids have a number of interesting properties. They are nonflammable, have an extremely low, barely measurable vapor pressure, are usually environmentally friendly, have a large liquidus range and have very good solvent properties for numerous substances. In addition, ionic liquids also have, owing to their purely ionic structure, interesting electrochemical properties such as electrical conductivity, frequently accompanied by a high electrochemical stability. The selection of suitable cations and anions enables, for example, the solubility in water or organic solvents or the melting point to be determined largely freely.

The molecular variety of ionic liquids makes it possible for them to be used in many industrial fields of application. Examples are extraction (e.g. isolation and purification of industrial gases, isolation and purification of hydrocarbons in petrochemistry and in organic synthesis or the removal of toxic substances from wastewater), the sorption, drying, purification and storage of gases (e.g. in sorption air conditioning units), use as solvents (e.g. for organic synthesis), the immobilization of catalysts, use as lubricants, hydraulic fluid or antistatic additive, use as electrolyte (e.g. in electroplating, in fuel cells, capacitors, sensor and battery technology, metal upgrading, photovoltaics or in electrochromic components), use as electroelastic material (e.g. in actuators), use for the transport or storage of heat (e.g. thermofluids or PCM media) or use as special analytical material (e.g. matrix materials, solvents for Karl-Fischer titration or media for protein crystallization or electrophoresis).

However, the use of ionic liquids is frequently not possible or possible for only a limited time despite their advantageous properties, for example in the abovementioned fields of application. Reasons are generally undesirable interactions between the ionic liquid and the materials brought into contact with it, for example influencing of the course of chemical reactions or corrosion of industrial plants. Such interactions can occur, depending on the ionic liquid used and/or the preparative process used, either only after a particular use time or right at the beginning of use and/or can occur to an increased extent with increasing use time.

It was an object of the present invention to provide a process by means of which those properties of ionic liquids which limit their use can be decreased. In this way, ionic liquids should be provided with those properties which are necessary for advantageous use or these properties of ionic liquids should be restored. Furthermore, the use of ionic liquids in fields of application for which they were previously unsuitable and/or longer operating lives of ionic liquids for use in industrial processes and/or their reuse should be made possible.

It has now been found that the cause of the abovementioned undesirable properties of ionic liquids is frequently contamination of the ionic liquids with small amounts of acidic and/or basic compounds. These impurities can be caused by the preparative process or else, for example, by decomposition reactions of the ionic liquids during storage or thermal stress. Such contaminated ionic liquids generally have an acidic or basic pH.

The present invention therefore provides a process for the work-up of an ionic liquid (IL) selected from among compounds of the general formula (I),

[A]⁺(1/n)*[Y]ⁿ⁻ (I)

where [A]⁺ is a quaternary ammonium cation and (1/n)*[Y]ⁿ⁻ is one anion equivalent of an n-fold charged anion,
where the ionic liquid (IL) has an acidic or basic pH,
which comprises adding a conjugate acid-base pair selected from among compounds of the formula (II),

[A]⁺[X]⁻(II)

where [A]⁺ has the meaning given for the ionic liquid and [X]⁻ is the conjugate base, to the ionic liquid (IL).

For the purposes of the present patent application, ionic liquids are organic salts which are liquid at temperatures below 180° C. The ionic liquids preferably have a melting point of less than 180° C. The melting point is more preferably in the range from −50° C. to 150° C., particularly preferably in the range from −20° C. to 120° C. and very particularly preferably below 100° C.

Ionic liquids which are present in the liquid state at room temperature are described, for example, by K. N. Marsh et al., Fluid Phase Equilibria 219 (2004), 93-98, and J. G. Huddleston et al., Green Chemistry 2001, 3, 156-164.

Both cations and anions are present in the ionic liquid. It is possible here for a proton or an alkyl radical to be transferred from the cation to the anion within the ionic liquid, resulting in two uncharged molecules. An equilibrium of anions, cations and uncharged molecules formed therefrom can thus be present in the ionic liquid used according to the invention.

For the purposes of the present invention, the expression “work-up” comprises the treatment of ionic liquids as are obtained from the production processes or by use in industrial processes with the aim of providing the ionic liquids with those properties which are required for advantageous use of the ionic liquids or restoring these properties. It is in any case an objective of the work-up to increase the operating lives of ionic liquids in industrial processes and/or to allow their reuse.

The expression “pH of ionic liquids” refers, for the purposes of the present invention, to the pH of an aqueous solution of an ionic liquid and not to the pH of the ionic liquid as such. Accordingly, the expression “acidic” in the context of ionic liquids is used for compounds whose aqueous solution has a pH of less than 7. The expression “basic” is used in the context of ionic liquids for compounds whose aqueous solution has a pH of greater than 7. To determine the “pH of ionic liquids”, 1 ml of the ionic liquid was in each case diluted with water to a total volume of 10 ml and the pH of the aqueous solution obtained in this way was determined by means of a glass electrode.

For the purposes of the present invention, the expression “conjugate acid-base pair” refers to a charge-neutral compound which formally comprises an anion (“conjugate base”) and a cation (“conjugate acid”). The ratio of basicity of the conjugate base and acidity of the conjugate acid determines the basic or acidic properties of the conjugate acid-base pair, i.e. if, for example, the basicity of the conjugate base predominates, the conjugate acid-base pair is basic.

For the purposes of the present invention, the expression “alkyl” comprises straight-chain or branched alkyl. Preference is given to straight-chain or branched C₁-C₃₀-alkyl, in particular C₁-C₁₈-alkyl and very particularly preferably C₁-C₄-alkyl. Examples of alkyl groups are, in particular, methyl, ethyl, n-propyl, 1-methylethyl (isopropyl), n-butyl, 2-methylpropyl (isobutyl), 1-methylpropyl (sec-butyl), 1,1-dimethylethyl (tert-butyl), n-pentyl, isopentyl, 1-methylbutyl, tert-pentyl, neopentyl, n-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, n-heptyl, n-octyl, 1-methylheptyl, 2-ethylhexyl, 2,4,4-trimethyl-pentyl, 1,1,3,3-tetramethylbutyl, n-nonyl, n-decyl, n-undecyl, n-dodecyl, n-tridecyl, n-tetradecyl, n-pentadecyl, n-hexadecyl, n-heptadecyl, n-octadecyl and n-eicosyl. Examples of C₁-C₄-alkyl are, in particular, methyl, ethyl, n-propyl, isopropyl, n-butyl and tert-butyl.

The expression alkyl also comprises alkyl radicals whose carbon chain can be interrupted by one or more nonadjacent heteroatoms or heteroatom-comprising groups which are preferably selected from among —O—, —S—, —NR^{E}—, —PR^{E}—, —SiR^{E}R^{EE} and/or —SO₂—. R^{E} is preferably H, alkyl, cycloalkyl, heterocycloalkyl, aryl or hetaryl. R^{EE} is preferably H, alkyl, cycloalkyl, heterocycloalkyl or aryl.

Examples of alkyl radicals whose carbon chains can be interrupted by one or two nonadjacent heteroatoms —O— are the following:

methoxymethyl, diethoxymethyl, 2-methoxyethyl, 2-ethoxyethyl, 2-propoxyethyl, diethoxyethyl, 2-butoxyethyl, 2-octyloxyethyl, 2-methoxypropyl, 3-methoxypropyl, 3-ethoxypropyl, 3-propoxypropyl, 2-isopropoxyethyl, 2-butoxypropyl, 3-butoxypropyl, 4-methoxybutyl, 4-ethoxybutyl, 4-propoxybutyl, 6-methoxyhexyl, 3,6-dioxaheptyl (5-methoxy-3-oxapentyl), 3,6-dioxaoctyl (7-methoxy-4-oxaheptyl), 4,8-dioxanonyl (7-methoxy-4-oxaheptyl), 3,7-dioxaoctyl, 3,7-dioxanonyl, 4,7-dioxaoctyl, 4,7-dioxa-nonyl, 2- and 4-butoxybutyl, 4,8-dioxadecyl, 9-ethoxy-5-oxanonyl.

Examples of alkyl radicals whose carbon chains can be interrupted by three or more nonadjacent heteroatoms —O— are oligooxyalkylenes and polyoxyalkylenes, i.e. compounds having repeating units which are preferably selected from among (CH₂CH₂O)_{X1}, (CH(CH₃)CH₂O)ₓ₂ and ((CH₂)₄O)ₓ₃ in which x1, x2 and x3 are each, independently of one another, an integer from 3 to 100, preferably from 3 to 80. The sum of x1, x2 and x3 is an integer from 3 to 300, in particular from 3 to 100. In polyoxy-alkylenes which have two or three different types of repeating units, the order is immaterial, i.e. the repeating units can be randomly distributed, alternate or be arranged in blocks. Examples are 3,6,9-trioxadecyl, 3,6,9-trioxaundecyl, 3,6,9-trioxa-dodecyl, 4,8,12-trioxamidecyl (11-methoxy-4,8-dioxaundecyl), 4,8,12-trioxatetradecyl, 14-methoxy-5,10-dioxatetradecyl, 5,10,15-trioxaheptadecyl, 3,6,9,12-tetraoxamidecyl, 3,6,9,12-tetraoxatetradecyl, 4,8,12,16-tetraoxaheptadecyl (15-methoxy-4,8,12-trioxa-pentadecyl), 4,8,12,16-tetraoxaoctadecyl and the like.

Examples of alkyl radicals whose carbon chains can be interrupted by one or more, e.g. 1, 2, 3, 4 or more than 4, nonadjacent heteroatoms —S— are the following:

butylthiomethyl, 2-methylthioethyl, 2-ethylthioethyl, 2-propylthioethyl, 2-butylthioethyl, 2-dodecylthioethyl, 3-methylthiopropyl, 3-ethylthiopropyl, 3-propylthiopropyl, 3-butylthiopropyl, 4-methylthiobutyl, 4-ethylthiobutyl, 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiamidecyl and 3,6,9,12-tetrathiatetradecyl.

Examples of alkyl radicals whose carbon chains are interrupted by one or two nonadjacent heteroatom-comprising groups —NR^{E}— are the following:

2-monomethylaminoethyl and 2-monoethylaminoethyl, 2-dimethylaminoethyl, 3-methylaminopropyl, 2- and 3-dimethylaminopropyl, 3-monoisopropylaminopropyl, 2- and 4-monopropylaminobutyl, 2- and 4-dimethylaminobutyl, 6-methylaminohexyl, 6-dimethylaminohexyl, 6-methyl-3,6-diazaheptyl, 3,6-dimethyl-3,6-diazaheptyl, 3,6-diazaoctyl and 3,6-dimethyl-3,6-diazaoctyl.

Examples of alkyl radicals whose carbon chains can be interrupted by three or more nonadjacent heteroatom-comprising groups —NR^{E}— also include oligoalkylenimines and polyalkylenimines. What has been said above for the polyoxyalkylenes applies analogously to polyalkylenimines, with the oxygen atom being replaced in each case by a group NR^{E}, where R^{a} is preferably H or C₁-C₄-alkyl. Examples are 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazamidecyl, 3,6,9,12-tetramethyl-3,6,9,12-tetra-azamidecyl and the like.

Examples of alkyl radicals whose carbon chains are interrupted by one or more, e.g. 1 or 2, nonadjacent groups —SO₂— are 2-methylsulfonylethyl, 2-ethylsulfonylethyl, 2-propylsulfonylethyl, 2-isopropylsulfonylethyl, 2-butylsulfonylethyl, 2-methylsulfonyl-propyl, 3-methylsulfonylpropyl, 2-ethylsulfonylpropyl, 3-ethylsulfonylpropyl, 2-propyl-sulfonylpropyl, 3-propylsulfonylpropyl, 2-butylsulfonylpropyl, 3-butylsulfonylpropyl, 2-methylsulfonylbutyl, 4-methylsulfonylbutyl, 2-ethylsulfonylbutyl, 4-ethylsulfonylbutyl, 2-propylsulfonylbutyl, 4-propylsulfonylbutyl and 4-butylsulfonylbutyl.

The expression alkyl also comprises substituted alkyl radicals. Substituted alkyl groups can, depending on the length of the alkyl chain, have one or more (e.g. 1, 2, 3, 4, 5 or more than 5) substituents. These are preferably selected independently from among cycloalkyl, cycloalkyloxy, polycyclyl, polycyclyloxy, heterocycloalkyl, aryl, aryloxy, arylthio, hetaryl, halogen, hydroxy, SH, ═O, ═S, ═NR^{E}, COOH, carboxylate, SO₃H, sulfonate, NE¹E², nitro and cyano, where E¹ and E² are each, independently of one another, H, alkyl, cycloalkyl, heterocycloalkyl, aryl or hetaryl. Cycloalkyl, cycloalkyloxy, polycycloalkyl, polycycloalkyloxy, heterocycloalkyl, aryl and hetaryl substituents of the alkyl groups can in turn be unsubstituted or substituted; suitable substituents are those mentioned below for these groups.

What has been said above with regard to alkyl also applies in principle to the alkyl parts of alkoxy, alkylamino, dialkylamino, alkylthio (alkylsulfanyl), alkylsulfinyl, alkylsulfonyl, etc.

Suitable substituted alkyl radicals are the following: alkyl which is substituted by carboxy, e.g. carboxymethyl, 2-carboxyethyl, 3-carboxy-propyl, 4-carboxybutyl, 5-carboxypentyl, 6-carboxyhexyl, 7-carboxyheptyl, 8-carboxy-octyl, 9-carboxynonyl, 10-carboxydecyl, 12-carboxydodecyl and 14-carboxytetradecyl;

alkyl which is substituted by SO₃H, e.g. sulfomethyl, 2-sulfoethyl, 3-sulfopropyl, 4-sulfo-butyl, 5-sulfopentyl, 6-sulfohexyl, 7-sulfoheptyl, 8-sulfooctyl, 9-sulfononyl, 10-sulfo-decyl, 12-sulfododecyl and 14-sulfotetradecyl;
alkyl which is substituted by carboxylate, for example, alkoxycarbonylalkyl, e.g. methoxycarbonylmethyl, ethoxycarbonylmethyl, n-butoxycarbonylmethyl, 2-methoxy-carbonylethyl, 2-ethoxycarbonylethyl, 2-methoxycarbonylpropyl, 2-ethoxycarbonyl-propyl, 2-(n-butoxycarbonyl)propyl, 2-(4-n-butoxycarbonyl)propyl, 3-methoxy-carbonylpropyl, 3-ethoxycarbonylpropyl, 3-(n-butoxycarbonyl)propyl, 3-(4-n-butoxy-carbonyl)propyl, aminocarbonylalkyl, e.g. aminocarbonylmethyl, aminocarbonylethyl, aminocarbonylpropyl and the like, alkylaminocarbonylalkyl such as methylamino-carbonylmethyl, methylaminocarbonylethyl, ethylcarbonylmethyl, ethylcarbonylethyl and the like, or dialkylaminocarbonylalkyl such as dimethylaminocarbonylmethyl, dimethylaminocarbonylethyl, dimethylcarbonylpropyl, diethylaminocarbonylmethyl, diethylaminocarbonylethyl, diethylcarbonylpropyl and the like;
alkyl which is substituted by hydroxy, e.g. 2-hydroxyethyl, 2-hydroxypropyl, 3-hydroxypropyl, 3-hydroxybutyl, 4-hydroxybutyl, 2-hydroxy-2,2-dimethylethyl, 5-hydroxy-3-oxapentyl, 6-hydroxyhexyl, 7-hydroxy-4-oxaheptyl, 8-hydroxy-4-oxaoctyl, 8-hydroxy-3,6-dioxaoctyl, 9-hydroxy-5-oxanonyl, 11-hydroxy-4,8-dioxaundecyl, 11-hydroxy-3,6,9-trioxaundecyl, 14-hydroxy-5,10-dioxatetradecyl, 15-hydroxy-4,8,12-trioxapentadecyl and the like;
alkyl which is substituted by amino, e.g. 2-aminoethyl, 2-aminopropyl, 3-aminopropyl, 4-aminobutyl, 6-aminohexyl and the like;
alkyl which is substituted by cyano, e.g. 2-cyanoethyl, 3-cyanopropyl, 3-cyanobutyl and 4-cyanobutyl;
alkyl which is substituted by halogen as defined below, where part or all of the hydrogen atoms in the alkyl group can be replaced by halogen atoms, for example C₁-C₁₈-fluoroalkyl, e.g. trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl and the like, C₁-C₁₈-chloroalkyl, e.g. chloromethyl, dichloromethyl, trichloromethyl, 2-chloroethyl, 2- and 3-chloropropyl, 2-, 3- and 4-chlorobutyl, 1,1-dimethyl-2-chloroethyl and the like, C₁-C₁₈-bromoalkyl, e.g. bromoethyl, 2-bromoethyl, 2- and 3-bromopropyl and 2-, 3- and 4-bromobutyl and the like;
alkyl which is substituted by nitro, e.g. 2-nitroethyl, 2- and 3-nitropropyl and 2-, 3- and 4-nitrobutyl and the like;
alkyl which is substituted by amino, e.g. 2-aminoethyl, 2-aminopropyl, 3-aminopropyl, 4-aminobutyl, 6-aminohexyl and the like;
alkyl which is substituted by cycloalkyl, e.g. cyclopentylmethyl, 2-cyclopentylethyl, 3-cyclopentylpropyl, cyclohexylmethyl, 2-cyclohexylethyl, 3-cyclohexylpropyl and the like;
alkyl which is substituted by ═O (oxo group), e.g. 2-oxopropyl, 2-oxobutyl, 3-oxobutyl, 1-methyl-2-oxopropyl, 2-oxopentyl, 3-oxopentyl, 1-methyl-2-oxobutyl, 1-methyl-3-oxobutyl, 2-oxohexyl, 3-oxohexyl, 4-oxohexyl, 2-oxoheptyl, 3-oxoheptyl, 4-oxoheptyl, 4-oxoheptyl and the like;
alkyl which is substituted by ═S (thioxo group), e.g. 2-thioxopropyl, 2-thioxobutyl, 3-thioxobutyl, 1-methyl-2-thioxopropyl, 2-thioxopentyl, 3-thioxopentyl, 1-methyl-2-thioxobutyl, 1-methyl-3-thioxobutyl, 2-thioxohexyl, 3-thioxohexyl, 4-thioxohexyl, 2-thioxoheptyl, 3-thioxoheptyl, 4-thioxoheptyl, 4-thioxoheptyl and the like;
alkyl which is substituted by ═NR^{E-}, preferably one in which R^{E} is H or C₁-C₄-alkyl, e.g. 2-iminopropyl, 2-iminobutyl, 3-iminobutyl, 1-methyl-2-iminopropyl, 2-iminopentyl, 3-iminopentyl, 1-methyl-2-iminobutyl, 1-methyl-3-iminobutyl, 2-iminohexyl, 3-imino-hexyl, 4-iminohexyl, 2-iminoheptyl, 3-iminoheptyl, 4-iminoheptyl, 4-iminoheptyl, 2-methyliminopropyl, 2-methyliminobutyl, 3-methyliminobutyl, 1-methyl-2-methyl-iminopropyl, 2-methyliminopentyl, 3-methyliminopentyl, 1-methyl-2-methyliminobutyl, 1-methyl-3-methyliminobutyl, 2-methyliminohexyl, 3-methyliminohexyl, 4-methyl-iminohexyl, 2-methyliminoheptyl, 3-methyliminoheptyl, 4-methyliminoheptyl, 4-methyl-iminoheptyl, 2-ethyliminopropyl, 2-ethyliminobutyl, 3-ethyliminobutyl, 1-methyl-2-ethyl-iminopropyl, 2-ethyliminopentyl, 3-ethyliminopentyl, 1-methyl-2-ethyliminobutyl, 1-methyl-3-ethyliminobutyl, 2-ethyliminohexyl, 3-ethyliminohexyl, 4-ethyliminohexyl, 2-ethyliminoheptyl, 3-ethyliminoheptyl, 4-ethyliminoheptyl, 4-ethyliminoheptyl, 2-propyliminopropyl, 2-propyliminobutyl, 3-propyliminobutyl, 1-methyl-2-propylimino-propyl, 2-propyliminopentyl, 3-propyliminopentyl, 1-methyl-2-propyliminobutyl, 1-methyl-3-propyliminobutyl, 2-propyliminohexyl, 3-propyliminohexyl, 4-propyl-iminohexyl, 2-propyliminoheptyl, 3-propyliminoheptyl, 4-propyliminoheptyl, 4-propyliminoheptyl and the like.

Alkyl which is substituted by aryl (“arylalkyl”) has at least one unsubstituted or substituted aryl group as defined below. Suitable substituents on the aryl group are those mentioned below. The alkyl group in “arylalkyl” can bear at least one further substituent as defined above and/or be interrupted by one or more nonadjacent heteroatoms or heteroatom-comprising groups selected from among —O—, —S—, —NR^{E}- and/or —SO₂—. Arylalkyl is preferably phenyl-C₁-C₁₀-alkyl, particularly preferably phenyl-C₁-C₄-alkyl, e.g. benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl, 4-phenbut-2-yl, 1-(phenmeth)eth-1-yl, 1-(phenmethyl)-1-(methyl)eth-1-yl or -(phenmethyl)-1-(methyl)prop-1-yl; preferably benzyl and 2-phenethyl.

Alkoxy is an alkyl group bound via an oxygen atom. Examples of alkoxy are: methoxy, ethoxy, n-propoxy, 1-methylethoxy, butoxy, 1-methylpropoxy, 2-methylpropoxy, 1,1-dimethylethoxy, n-pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethyl-propoxy, 1,2,2-trimethylpropoxy, 1-ethyl-1-methylpropoxy or 1-ethyl-2-methylpropoxy, hexoxy and R^{A}O—(CH₂CH₂CH₂CH₂O)ₙ—CH₂CH₂CH₂CH₂O— where R^{A} is H or C₁-C₄-alkyl, preferably H, methyl or ethyl and n is from 0 to 10, preferably from 0 to 3.

Alkylthio (alkylsulfanyl) is an alkyl group bound via a sulfur atom. Examples of alkylthio are methylthio, ethylthio, propylthio, butylthio, pentylthio and hexylthio.

Alkylsulfinyl is an alkyl group bound via an S(═O)group.

Alkylsulfonyl is an alkyl group bound via an S(═O)₂group.

The expression “alkenyl” comprises, for the purposes of the present invention, straight-chain and branched alkenyl groups which, depending on the chain length, can have one or more double bonds (e.g. 1, 2, 3, 4 or more than 4). Preference is given to C₂-C₁₈₋, particularly preferably C₂-C₁₂-alkenyl groups. The expression “alkenyl” also comprises substituted alkenyl groups which can bear one or more (e.g. 1, 2, 3, 4, 5 or more than 5) substituents. Suitable substituents are, for example, selected from among ═O, ═S, ═NR^{E}, cycloalkyl, cycloalkyloxy, polycyclyl, polycyclyloxy, heterocycloalkyl, aryl, aryloxy, arylthio, hetaryl, halogen, hydroxy, SH, COOH, carboxylate, SO₃H, sulfonate, alkylsulfinyl, alkylsulfonyl, NE³E⁴, nitro and cyano, where E³ and E⁴ are each, independently of one another H, alkyl, cycloalkyl, heterocycloalkyl, aryl or hetaryl.

The expression “alkenyl” also comprises alkenyl radicals whose carbon chain can be interrupted by one or more nonadjacent heteroatoms or heteroatom-comprising groups which are preferably selected from among —O—, —S—, —NR^{E}- and/or —SO₂—. Alkenyl is then, for example ethenyl (vinyl), 1-propenyl, 2-propenyl, 1-methylethenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like.

The expression “cycloalkyl” comprises, for the purposes of the present invention, both unsubstituted and substituted monocyclic saturated hydrocarbon groups which in general have from 3 to 12 ring carbons, preferably C₃-C₁₂-cycloalkyl groups such as cyclopropyl, cyclobutyl, cyclopentyl, cyclohexyl, cycloheptyl, cyclooctyl, cyclononyl, cyclodecyl, cycloundecyl or cyclododecyl, in particular C₅-C₁₂-cycloalkyl. Suitable substituents are generally selected from among alkyl, the substituents mentioned above for alkyl groups, alkoxy and alkylthio. Substituted cycloalkyl groups can have one or more (e.g. 1, 2, 3, 4, 5 or more than 5) substituents, with in the case of halogen the cycloalkyl radical being partially or fully substituted by halogen.

Examples of cycloalkyl groups are cyclopentyl, 2- and 3-methylcyclopentyl, 2- and 3-ethylcyclopentyl, chloropentyl, dichloropentyl, dimethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, chlorohexyl, dimethylcyclohexyl, diethylcyclohexyl, methoxy-cyclohexyl, dimethoxycyclohexyl, diethoxycyclohexyl, butoxycyclohexyl, methylthio-cyclohexyl, chlorocyclohexyl, dichlorocyclohexyl, cycloheptyl, 2-, 3- and 4-methylcyclo-heptyl, 2-, 3- and 4-ethylcycloheptyl, 3- and 4-propylcycloheptyl, 3- and 4-isopropyl-cycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethyl-cyclooctyl, 3-, 4- and 5-propylcyclooctyl, partially fluorinated cycloalkyl and perfluorinated cycloalkyl of the formula CₙF_{2(n-a)-(1-b)}H₂a-b where n=5 to 12, 0 <=a <=n and b=0 or 1.

Cycloalkyloxy is a cycloalkyl group as defined above bound via oxygen.

The expression “cycloalkenyl” comprises unsubstituted and substituted, monounsaturated or doubly unsaturated hydrocarbon groups having from 3 to 5, up to 8, up to 12, preferably from 5 to 12, ring carbons, e.g. cyclopent-1-en-1-yl, cyclopent-2-en-1-yl, cyclopent-3-en-1-yl, cyclohex-1-en-1-yl, cyclohex-2-en-1-yl, cyclohex-3-en-1-yl, cyclohexa-2,5-dien-1-yl and the like. Suitable substituents are those mentioned above for cycloalkyl.

Cycloalkenyloxy is a cycloalkenyl group as defined above bound via oxygen.

For the purposes of the present invention, the expression “polycyclyl” comprises in the broadest sense compounds which comprise at least two rings, regardless of how these rings are linked. The rings can be carbocyclic and/or heterocyclic rings. The rings can be saturated or unsaturated. The rings can be linked via a single or double bond (“multiring compounds”), bound by fusion (“fused ring systems”) or bridged (“bridged ring systems”, “cage compounds”). Preferred polycyclic compounds are bridged ring systems and fused ring systems. Fused ring systems can be aromatic, hydroaromatic and cyclic compounds joined by fusion (fused). Fused ring systems comprise two, three or more than three rings. Depending on the way in which they are joined, a distinction is made in the case of fused ring systems between ortho-fusion, i.e. each ring shares an edge or two atoms with each neighboring ring, and peri-fusion in which a carbon atom belongs to more than two rings. Among fused ring systems, preference is given to ortho-fused ring systems. For the purposes of the present invention, bridged ring systems include those which are not included under multiring systems nor under fused ring systems and in which at least two ring atoms belong to at least two different rings. In the case of bridged ring systems, a distinction is made between bicyclo, tricyclo, tetracyclo compounds, etc., which comprise two, three, four, etc. rings according to the number of ring opening reactions which are formally required to obtain an open-chain compound. The expression “bicycloalkyl” comprises bicyclic hydrocarbon radicals which preferably have from 5 to 10 carbon atoms, e.g. bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like. The expression “bicycloalkenyl” comprises monounsaturated, bicyclic hydrocarbon radicals which preferably have from 5 to 10 carbon atoms, e.g. bicyclo[2.2.1]hept-2-en-1-yl.

The expression “aryl” comprises, for the purposes of the present invention, aromatic hydrocarbon radicals which have one or more rings and can be unsubstituted or substituted. The expression aryl generally refers to hydrocarbon radicals having from 6 to 10, up to 14, up to 18, preferably from 6 to 10, ring carbons. The term aryl preferably refers to unsubstituted or substituted phenyl, naphthyl, anthracenyl, phenanthrenyl, naphthacenyl, chrysenyl, pyrenyl, etc., and particularly preferably phenyl or naphthyl. Substituted aryls can, depending on the number and size of their ring systems, have one or more (e.g. 1, 2, 3, 4, 5 or more than 5) substituents. These are preferably selected independently from among alkyl, alkoxy, cycloalkyl, cycloalkyloxy, heterocycloalkyl, aryl, aryloxy, arylthio, hetaryl, halogen, hydroxy, SH, alkylthio, alkylsulfinyl, alkylsulfonyl, COOH, carboxylate, SO₃H, sulfonate, NE⁵E⁶, nitro and cyano, where E⁵ and E⁶ are each, independently of one another, H, alkyl, cycloalkyl, cycloalkyloxy, polycyclylyl, polycyclyloxy, heterocycloalkyl, aryl, aryloxy or hetaryl. Aryl is particularly preferably phenyl which, if it is substituted, can generally bear 1, 2, 3, 4 or 5, preferably 1, 2 or 3, substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl, 2,4,6-tri-tert-butylphenyl and 2-, 3-, 4-dodecylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxy-phenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl, 2-, 3- and 4-butoxyphenyl, 2-, 3-, 4-hexyloxy-phenyl; 2-, 3-, 4-chlorophenyl, 2,4-, 2,5-, 3,5- and 2,6-dichlorophenyl, trichlorophenyl, 2-, 3-, 4-fluorophenyl, 2,4-, 2,5-, 3,5- and 2,6-difluorophenyl, trifluorophenyl, e.g. 2,4,6-trifluorophenyl, tetrafluorophenyl, pentafluorophenyl, 2-, 3- and 4-cyanophenyl; 2-nitrophenyl, 4-nitrophenyl, 2,4-dinitrophenyl, 2,6-dinitrophenyl; 4-dimethylamino-phenyl; 4-acetylphenyl; methoxyethylphenyl, ethoxymethylphenyl; methylthiophenyl, isopropylthiophenyl or tert-butylthiophenyl; methylnaphthyl; isopropylnaphthyl or ethoxynaphthyl. Examples of substituted aryl in which two substituents bound to adjacent carbon atoms of the aryl ring form a fused ring or fused ring system are indenyl and fluorenyl.

The expression “aryloxy” refers, for the purposes of the present invention, to aryl bound via an oxygen atom.

The expression “arylthio” refers, for the purposes of the present invention, to aryl bound via a sulfur atom.

The expression “heterocycloalkyl” comprises, for the purposes of the present invention, nonaromatic, unsaturated or fully saturated, cycloaliphatic groups which generally have from 5 to 8 ring atoms, preferably 5 or 6 ring atoms, in which 1, 2 or 3 of the ring carbons are replaced by heteroatoms selected from among oxygen, nitrogen, sulfur and a group —NR^{E}— and which are unsubstituted or substituted by one or more, for example 1, 2, 3, 4, 5 or 6, C₁-C₆-alkyl groups. Examples of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothienyl, dihydrothienyl, tetrahydrofuranyl, dihydrofuranyl, tetrahydropyranyl, 1,2-oxazolin-5-yl, 1,3-oxazolin-2-yl and dioxanyl. Nitrogen-comprising heterocycloalkyl can in principle be bound either via a carbon atom or via a nitrogen atom.

The expression “heteroaryl (hetaryl)” comprises, for the purposes of the present invention, unsubstituted or substituted, heteroaromatic groups which have one or more rings and generally from 5 to 14 ring atoms, preferably 5 or 6 ring atoms, and in which 1, 2 or 3 of the ring carbons are replaced by one, two, three or four heteroatoms, selected from among O, N, —NR^{E}— and S, e.g. furyl, thienyl, oxazolyl, isoxazolyl, thiazolyl, isothiazolyl, benzofuranyl, benzthiazolyl, benzimidazolyl, pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, where these heterocycloaromatic groups can, if they are substituted, generally bear 1, 2 or 3 substituents. The substituents are generally selected from among C₁-C₆-alkyl, C₁-C₆-alkoxy, hydroxy, carboxy, halogen and cyano.

5- to 7-membered nitrogen-comprising heterocycloalkyl or heteroaryl radicals, which optionally comprise further heteroatoms, are, for example, pyrrolyl, pyrazolyl, imidazolyl, triazolyl, pyrrolidinyl, pyrazolinyl, pyrazolidinyl, imidazolinyl, imidazolidinyl, pyridinyl, pyridazinyl, pyrimidinyl, pyrazinyl, triazinyl, piperidinyl, piperazinyl, oxazolyl, isooxazolyl, thiazolyl, isothiazolyl, indolyl, quinolinyl, isoquinolinyl or quinaldinyl, which can be unsubstituted or substituted as described above.

Halogen is fluorine, chlorine, bromine or iodine.

For the purposes of the present invention, carboxylate and sulfonate are preferably a derivative of a carboxylic acid function or a sulfonic acid function, in particular a metal carboxylate or sulfonate, a carboxylic ester or sulfonic ester function or a carboxamide or sulfonamide function. These include, for example, the ester with C₁-C₄-alkanols, such as methanol, ethanol, n-propanol, isopropanol, n-butanol, sec-butanol and tert-butanol.

The expression “acyl” refers, for the purposes of the present invention, to alkanoyl, hetaroyl or aroyl groups which generally have from 1 to 11, preferably from 2 to 8, carbon atoms, for example the formyl, acetyl, propanoyl, butanoyl, pentanoyl, hexanoyl, heptanoyl, 2-ethylhexanoyl, 2-propylheptanoyl, benzoyl or naphthoyl group.

The radicals E¹ and E², E³ and E⁴, E⁵ and E⁶ are selected independently from among H, alkyl, cycloalkyl and aryl. The groups NE¹E², NE³E⁴ and NE⁵E⁶ are preferably N,N-dimethylamino, N,N-diethylamino, N,N-dipropylamino, N,N-diisopropylamino, N,N-di-n-butylamino, N,N-di-tert-butylamino, N,N-dicyclohexylamino or N,N-diphenyl-amino.

Compounds which are suitable for forming the cations [A]⁺of ionic liquids are described, for example, in DE 102 02 838 A1. These compounds comprise at least one nitrogen atom, particularly preferably from 1 to 10 nitrogen atoms, in particular from 1 to 5 nitrogen atoms, very particularly preferably from 1 to 3 nitrogen atoms and especially 1 or 2 nitrogen atoms. These compounds can comprise further heteroatoms such as oxygen, sulfur or phosphorus atoms.

In the synthesis of the ionic liquids, an ammonium ion can firstly be generated by quaternization of the nitrogen atom of an amine or a nitrogen heterocycle. Quaternization can be effected, for example, by protonation or alkylation of the nitrogen atom. Depending on the protonating or alkylating agent used, salts having different anions are obtained. In cases in which it is not possible to form the desired anion in the quaternization, this can be effected in a further synthesis step. Starting from, for example, an ammonium halide, the halide can be reacted with a Lewis acid to form a complex anion of halide and Lewis acid. As an alternative, replacement of a halide ion by the desired anion is possible. This can be achieved by addition of a metal salt with precipitation of the metal halide formed, by means of an ion exchanger or by displacement of the halide ion by a strong acid (with liberation of the hydrohalic acid). Suitable processes are described, for example, in Angew. Chem. 2000, 112, pp. 3926-3945, and the references cited therein.

The above-described process is particularly suitable for the work-up of acidic ionic liquids as are obtained, for example, by preparation from the corresponding halides, as a result of thermal stress or partial hydrolysis in industrial processes or as a result of storage. Basic conjugate acid-base pairs, i.e. compounds of the formula (II) which in aqueous solution have a pH of greater than 7, are particularly suitable for the work-up of such ionic liquids. The pH of the compound of the formula (II) is determined in aqueous solution.

In a specific embodiment of the present invention, ionic liquids (IL) which have a pH in the range from 1 to 6.9 and in particular in the range from 2 to 6.5 and in which the conjugate acid-base pair of the formula (II) is selected from among basic compounds are therefore worked up by the process of the invention.

Particularly suitable basic acid-base pairs of the formula (II) are those in which the conjugate base [X]⁻ is an anion of the formula ⁻OR¹, where R¹ is alkyl or cycloalkyl. R¹ is preferably C₁-C₄-alkyl and particularly preferably methyl, ethyl, n-propyl, 1-methylethyl, n-butyl, 2-methylpropyl or 2,2-dimethylethyl.

Furthermore, it has been found to be advantageous for the neutralization of the ionic liquid to form, as neutralization product, a compound of the formula X-H which can be separated off from the ionic liquid by conventional separation processes, in particular by distillation.

In the process of the invention, the anion [Y]ⁿ⁻ of the ionic liquids of the formula (I) is preferably selected from among compounds of the formulae (R^{a})SO₃⁻, (R^{a})SO₂⁻, (R^{a})PO₃²⁻, (R^{a})(R^{b})PO₂⁻, (R^{a})PO₂²⁻, (R^{a})(R^{b})PO⁻, (R^{a})BO₂²⁻, (R^{a})(R^{b})BO⁻, (R^{a})(R^{b})(R^{c})(R^{d})B⁻, (R^{a})CO₂⁻, (R^{a})SiO₃³⁻, (R^{a})(R^{b})SiO₂²⁻ and (R^{a})(R^{b})(R^{c})SiO⁻, where the radicals R^{a}, R^{b}, R^{c} and R^{d} are each, independently of one another, OH, alkyl, alkoxy, aryl, aryloxy, cycloalkyl, cycloalkoxy, heterocycloalkyl, heterocycloalkyloxy, heteroaryl or heteroaryloxy and in particular alkyl, alkoxy, aryl or aryloxy.

Ionic liquids which have one of the anions [Y]ⁿ⁻ mentioned as preferred are generally obtained from their production process with a pH of less than 7, frequently from 2 to 6.5, and/or are subject, for example during storage or in the case of thermal stress, to decomposition reactions which lead to a reduction in the pH. It has now been found that this property of ionic liquids frequently has an adverse effect over the course of time on the processes in which the ionic liquids are advantageously used. This results in limited operating lives of the ionic liquids and thus increased costs.

[Y]ⁿ⁻ in the compounds of the formula (I) is particularly preferably selected from among compounds of the formulae (R^{a})SO₃⁻, (R^{a})SO₂⁻, (R^{a})PO₃²⁻, (R^{a})(R^{b})PO₂⁻, (R^{a})PO₂²⁻, (R^{a})(R^{b})PO⁻and (R^{a})PO²⁻, where R^{a} and R^{b} are each, independently of one another, OH, alkyl, alkoxy, aryl, aryloxy, cycloalkyl, cycloalkoxy, heterocycloalkyl, heterocyclo-alkyloxy, heteroaryl or heteroaryloxy and in particular alkyl, alkoxy, aryl or aryloxy.

[Y]ⁿ⁻ in the compounds of the formula (I) is very particularly preferably selected from among compounds of the formulae (R^{a})SO₃⁻and (R^{a})(R^{b})PO₂⁻, especially compounds of the formula (R^{a})SO₃⁻, where R^{a} and R^{b} are each, independently of one another, alkyl, alkoxy, aryl or aryloxy.

Preference is given to R^{a}, R^{b}, R^{c} and R^{d} in the anions [Y]ⁿ⁻ each being, independently of one another, OH, alkyl, alkoxy, aryl, aryloxy, cycloalkyl, cycloalkoxy, heterocycloalkyl, heterocycloalkyloxy, heteroaryl or heteroaryloxy, particularly preferably alkyl, alkoxy, aryl or aryloxy and very particularly preferably C₁-C₄-alkyl, C₁-C₄-alkoxy or phenyl, where phenyl is unsubstituted or bears 1, 2 or 3 substituents selected from among halogen, CN, NO₂, C₁-C₄-alkyl, C₁-C₄-alkoxy and di(C₁-C₄-alkyl)amino. Preferred cations [A]⁺ are compounds which have a molar mass of less than 1000 g/mol, very particularly preferably less than 600 g/mol and in particular less than 400 g/mol.

Preferred cations [A]⁺ are also compounds which comprise at least one five- or six-membered heterocycle, in particular a five-membered heterocycle, which has at least one nitrogen atom and optionally an oxygen or sulfur atom, with particular preference being given to compounds which comprise at least one five- or six-membered heterocycle having one, two or three nitrogen atoms and a sulfur or oxygen atom, very particularly preferably those having two nitrogen atoms. Further preference is given to aromatic heterocycles.

Accordingly, an ionic liquid (IL) which has a heterocyclic cation [A]⁺ will be worked up in a preferred embodiment of the process of the invention.

For the purposes of the present invention, the term “heterocyclic” cation comprises both “heteroaromatic” cations and “partially or fully saturated heterocyclic cations”.

The term “heteroaromatic” cation comprises cations whose structure can be arrived at, for example, by quaternization of a ring nitrogen of a “hetaryl” compound as defined above. Examples of five- or six-membered heteroaromatic cations are pyrazolium, oxazolium, isoxazolium, thiazolium, isothiazolium, imidazolium, 1,2,4-oxadiazolium, 1,2,4-thiadiazolium, 1,3,4-oxadiazolium, 1,3,4-thiadiazolium, pyrrolium, 1,2,3-triazolium, 1,2,4-triazolium, pyridinium, pyridazinium, pyrimidinium, 2-pyrazinium, 1,3,5-triazinium and 1,2,4-triazinium.

The term “partially or fully saturated” heterocyclic cation comprises cations whose structure can be arrived at, for example, by quaternization of a ring nitrogen of a “heterocycloalkyl” compound as defined above. Examples of five- or six-membered saturated or partially unsaturated heterocyclic cations are pyrrolidinium, pyrazolidinium, oxazolidinium, isoxazolidinium, thiazolidinium, isothiazolidinium, imidazolidinium, 1,2,4-oxadiazolidinium, 1,2,4-thiadiazolidinium, 1,2,4-triazolidinium, 1,3,4-oxa-diazolidinium, 1,3,4-thiadiazolidinium, 1,3,4-triazolidinium, 2-pyrrolinium, 3-pyrrolinium, 2-isoxazolinium, 3-isoxazolinium, 4-isoxazolinium, 2-isothiazolinium, 3-isothiazolinium, 4-isothiazolinium, 2,3-dihydropyrazolium, 3,4-dihydropyrazolium, 4,5-dihydro-pyrazolium, 2,3-dihydrooxazolium, 3,4-dihydrooxazolium, piperidinium, hexahydro-pyridazinium, hexahydropyrimidinium, piperazinium, 1,3,5-hexahydrotriazinium or 1,2,4-hexahydrotriazinium.

The ionic liquid (IL) which is worked up according to the invention preferably comprises at least one cation selected from among the compounds of the formulae (IV.a) to (IV.v) shown below,

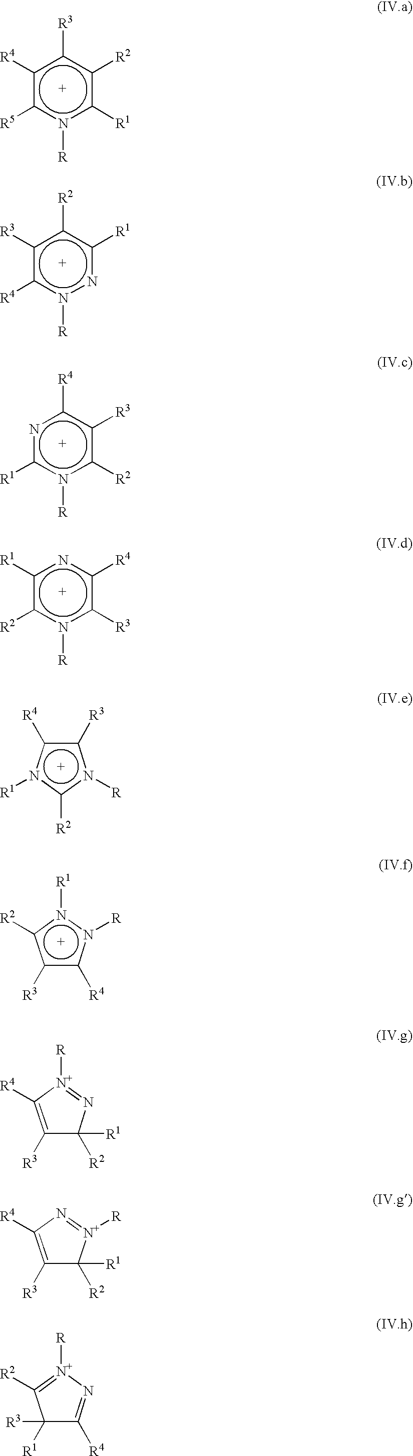

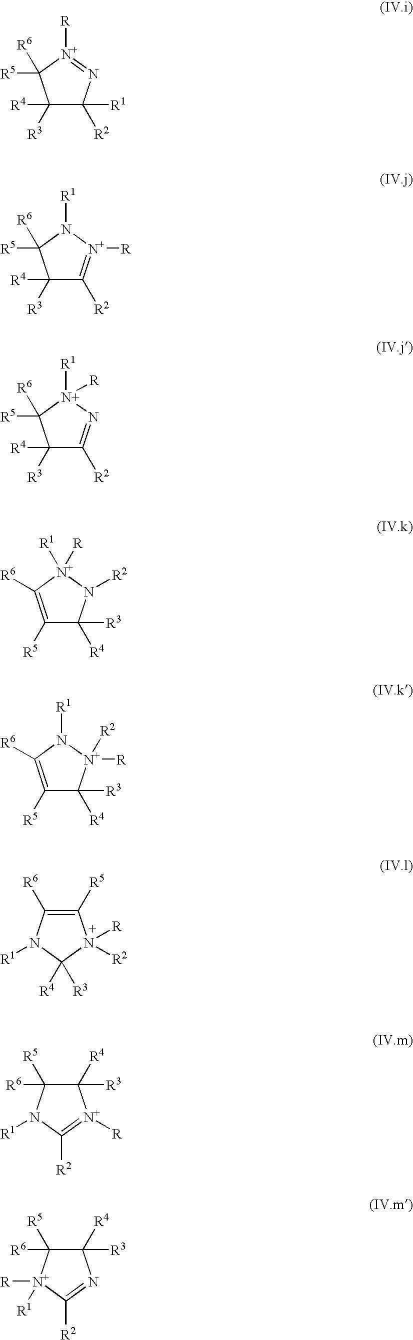

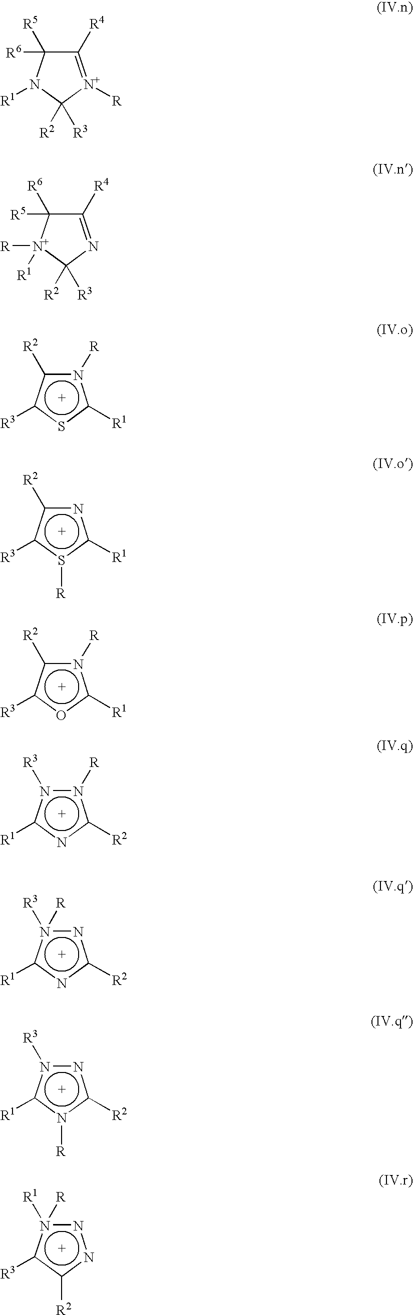

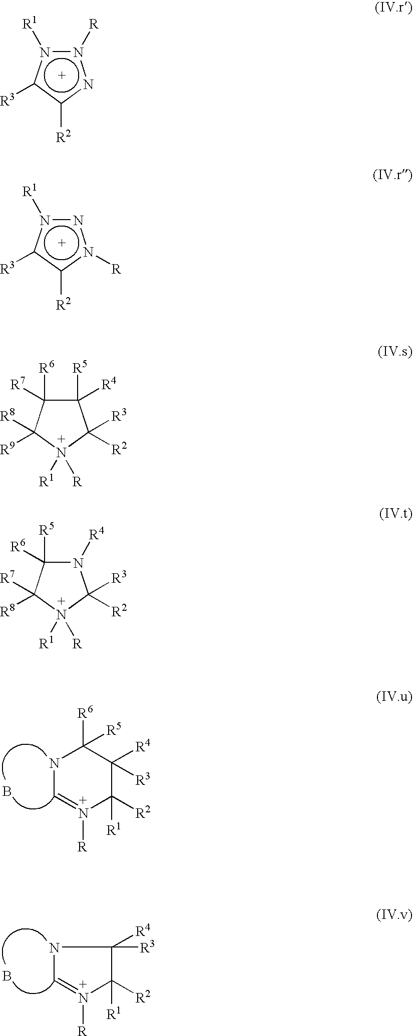

and oligomers comprising these structures, where
R is H, alkyl, alkenyl, cycloalkyl, cycloalkenyl, polycyclyl, heterocycloalkyl, aryl or heteroaryl;
radicals R¹, R², R³, R⁴, R⁵, R⁶, R⁷, R⁸ and R⁹ which are bound to a ring carbon are each, independently of one another, H, a sulfo group, COOH, carboxylate, sulfonate, acyl, alkoxycarbonyl, cyano, halogen, hydroxyl, SH, nitro, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, where E¹ and E² are each, independently of one another, H, alkyl, cycloalkyl, heterocycloalkyl, aryl or hetaryl,
radicals R¹, R², R³, R⁴, R⁵, R⁶, R⁷, R⁸ and R⁹ which are bound to a ring heteroatom are H, SO₃H, NE¹E², alkyl, alkoxy, alkenyl, cycloalkyl, cycloalkenyl, polycyclyl, heterocycloalkyl, aryl or heteroaryl, where E¹ and E² are each, independently of one another, H, alkyl, cycloalkyl, heterocycloalkyl, aryl or hetaryl, or
two adjacent radicals R¹ to R⁹ together with the ring atoms to which they are bound can also form at least one fused, saturated, unsaturated or aromatic ring or ring system having from 1 to 30 carbon atoms, where the ring or the ring system can have from 1 to 5 nonadjacent heteroatoms or heteroatom-comprising groups and the ring or the ring system can be unsubstituted or substituted,
where two geminal radicals R¹ to R⁹ can also together form ═O, ═S or ═NR^{b}, where R^{b} is H, alkyl, cycloalkyl, aryl or heteroaryl,
where, in the compounds of the formula (IV.u), R¹ and R³ or R³ and R⁵ can also together represent the second bond of a double bond between the ring atoms which bear these radicals,
B in the compounds of the formulae (IV.u) and (IV.v) together with the CN group to which it is bound forms a 4- to 8-membered, saturated or unsaturated or aromatic ring which is optionally substituted and/or can optionally have further heteroatoms or heteroatom-comprising groups and/or can comprise further fused saturated, unsaturated or aromatic carbocycles or heterocycles.

As regards the general meanings of the abovementioned radicals carboxylate, sulfonate, acyl, alkoxycarbonyl, halogen, NE¹E², alkyl, alkoxy, alkylthio, alkylsulfinyl, alkylsulfonyl, alkenyl, cycloalkyl, cycloalkyloxy, cycloalkenyl, cycloalkenyloxy, polycyclyl, polycyclyloxy, heterocycloalkyl, aryl, aryloxy or heteroaryl, reference is made to the full scope of what has been said above. Radicals R¹ to R⁹ which are bound to a carbon atom in the abovementioned formulae (IV) and have a heteroatom or a heteroatom-comprising group can also be bound directly via a heteroatom to the carbon atom.

If two adjacent radicals R¹ to R⁹ together with the ring atoms to which they are bound form at least one fused, saturated, unsaturated or aromatic ring or ring system having from 1 to 30 carbon atoms, where the ring or the ring system can have from 1 to 5 nonadjacent heteroatoms or heteroatom-comprising groups and the ring or the ring system can be unsubstituted or substituted, these radicals can together preferably be, as fused-on building blocks, 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The radical R in the compounds of the formulae IV.a to IV.v is preferably

unsubstituted C₁-C₁₈-alkyl such as methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl;
C₁-C₁₈-alkyl substituted by one or more hydroxy, halogen, phenyl, cyano, C₁-C₆-alkoxy-carbonyl and/or SO₃H groups, especially hydroxy-C₁-C₁₈-alkyl, e.g. 2-hydroxyethyl or 6-hydroxyhexyl; phenyl-C₁-C₁₈-alkyl, e.g. benzyl, 3-phenylpropyl; cyano-C₁-C₁₈-alkyl, e.g. 2-cyanoethyl; C₁-C₆-alkoxy-C₁-C₁₈-alkyl, e.g. 2-(methoxycarbonyl)ethyl, 2-(ethoxycarbonyl)ethyl or 2-(n-butoxycarbonyl)ethyl; C₁-C₁₈-fluoroalkyl, such as trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl; sulfo-C₁-C₁₈-alkyl, e.g. 3-sulfopropyl;
hydroxyethyloxyalkyl, radicals of oligoalkylene glycols and polyalkylene glycols such as polyethylene glycols and polypropylene glycols and oligomers thereof having from 2 to 100 units and an H or C₁-C₈-alkyl as end group, for example R^{A}O—(CHR^{B}—CH₂—O)ₙ—CHR^{B}—CH₂— where R^{A} and R^{B} is preferably H, methyl or ethyl and n is preferably from 0 to 3, in particular 3-oxa-butyl, 3-oxa-pentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl; and
C₂-C₆-alkenyl, such as vinyl or propenyl.

The radical R is particularly preferably linear C₁-C₁₈-alkyl, for example methyl, ethyl, 1-propyl, 1-butyl, 1-pentyl, 1-hexyl, 1-heptyl, 1-octyl, 1-decyl, 1-dodecyl, 1-tetradecyl, 1-hexadecyl, 1-octadecyl, very particularly preferably methyl, ethyl, 1-butyl or 1-octyl, or CH₃O—(CH₂CH₂O)ₙ—CH₂CH₂— or CH₃CH₂O—(CH₂CH₂O)ₘ—CH₂CH₂—, where m is from 0 to 3.

Preference is given to the radicals R¹ to R⁹ in the compounds of the formulae IV.a to IV.v each being, independently of one another, H, halogen, hydroxy, alkoxy, alkylthio, carboxyl, —COOH, sulfonate, CN, NO₂, acyl, alkoxycarbonyl, NE¹E², where E¹ and E² have one of the meanings given above,

C₁-C₁₈-alkyl which is unsubstituted or substituted and/or can be interrupted by at least one heteroatom or a heteroatom-comprising group,
C₂-C₁₈-alkenyl which is unsubstituted or substituted and/or can be interrupted by at least one heteroatom,
C₆-C₁₀-aryl which is unsubstituted or substituted,
C₅-C₁₂-cycloalkyl which is unsubstituted or substituted,
Polycyclyl which is unsubstituted or substituted,
C₅-C₁₂-cycloalkenyl which is unsubstituted or substituted,
heterocycloalkyl which has 5 or 6 ring atoms, where the ring has 1, 2 or 3 heteroatoms or heteroatom-comprising groups selected from among oxygen, nitrogen, sulfur and NR^{E} in addition to ring carbons, and is unsubstituted or substituted or
heteroaryl which has 5 to 10 ring atoms, where the ring has 1, 2 or 3 heteroatoms or heteroatom-comprising groups selected from among oxygen, nitrogen, sulfur and NR^{E} in addition to ring carbons and is unsubstituted or substituted.

Preference is likewise given to two adjacent radicals R¹ to R⁹ in the compounds of the formulae IV.a to IV.v together with the ring atoms to which they are bound forming at least one fused, saturated, unsaturated or aromatic ring or ring system having from 1 to 12 carbon atoms, where the ring or the ring system can have from 1 to 5 nonadjacent heteroatoms or heteroatom-comprising groups which are preferably selected from among oxygen, nitrogen, sulfur and NR^{E} and the ring or the ring system is unsubstituted or can be substituted by substituents which are preferably selected independently from among alkoxy, cycloalkyl, cycloalkoxy, polycyclyl, polycyclyloxy, heterocycloalkyl, aryl, aryloxy, arylthio, heteroaryl halogen, hydroxy, SH, ═O, ═S, ═NR^{E}, COOH, carboxylate, —SO₃H, sulfonate, NE¹E², nitro and cyano, where E¹ and E² are each, independently of one another, H, alkyl, cycloalkyl, heterocycloalkyl, aryl or hetaryl.

When R¹ to R⁹ in the compounds of the formulae IV.a to IV.v are alkoxy, then R¹ to R⁹ are preferably methoxy or ethoxy or R^{A}O—(CH₂CH₂CH₂CH₂O)ₙ—CH₂CH₂CH₂CH₂O—, where R^{A} and R^{B} are preferably H, methyl or ethyl and n is preferably from 0 to 3.

When R¹ to R⁹ in the compounds of the formulae IV.a to IV.v are acyl, these are preferably selected from among formyl and C₁-C₄-alkylcarbonyl, in particular formyl or acetyl.

When R¹ to R⁹ in the compounds of the formulae IV.a to IV.v are C₁-C₁₈-alkyl, these are preferably selected from among unsubstituted C₁-C₁₈-alkyl such as methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-9-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl;

C₁-C₁₈-haloalkyl, especially C₁-C₁₈-fluoroalkyl, for example trifluoromethyl, difluoro-methyl, fluoromethyl, pentafluoroethyl, heptafluoropropyl, heptafluoroisopropyl, nonafluorobutyl, nonofluoroisobutyl, undecylfluoropentyl, undecylisopentyl, C₆F₁₃, C₈F₁₇, C₁₀F₂₁, C₁₂F₂₅, especially C₁-C₁₈-chloroalkyl, such as chloromethyl, 2-chloroethyl, trichloromethyl, 1,1-dimethyl-2-chloroethyl;
amino-C₁-C₁₈-alkyl such as 2-aminoethyl, 2-aminopropyl, 3-aminopropyl, 4-aminobutyl, 6-aminohexyl,
C₁-C₆-alkylamino-C₁-C₁₈-alkyl such as 2-methylaminoethyl, 2-methylaminopropyl, 3-methylaminopropyl, 4-methylaminobutyl, 6-methylaminohexyl;
di(C₁-C₆-alkyl)-C₁-C₁₈-alkyl such as 2-dimethylaminoethyl, 2-dimethylaminopropyl, 3-dimethylaminopropyl, 4-dimethylaminobutyl, 6-dimethylaminohexyl, cyano-C₁-C₁₈-alkyl such as 2-cyanoethyl, 2-cyanopropyl, C₁-C₁₀-alkoxy-C₁-C₁₈-alkyl such as methoxymethyl, 2-methoxyethyl, 2-methoxypropyl, 3-methoxypropyl, 2-methoxyisopropyl, 4-methoxybutyl, 6-methoxyhexyl, 2-ethoxyethyl, 2-ethoxypropyl, 3-ethoxypropyl, 4-ethoxybutyl, 6-ethoxyhexyl, 2-isopropoxyethyl, 2-butoxyethyl, 2-butoxypropyl, 2-octyloxyethyl, 5-methoxy-3-oxapentyl, 8-methoxy-3,6-dioxaoctyl, 7-methoxy-4-oxaheptyl, 11-methoxy-4,8-dioxaundecyl, 9-methoxy-5-oxa-nonyl, 9-methoxy-5-oxanonyl, 14-methoxy-5,10-dioxatetradecyl, 5-ethoxy-3-oxapentyl, 8-ethoxy-3,6-dioxaoctyl, 7-ethoxy-4-oxaheptyl, 11-ethoxy-4,8-dioxaundecyl, 9-ethoxy-5-oxanonyl or 14-ethoxy-5,10-oxatetradecyl, 15-methoxy-4,8,12-trioxapentadecyl, 11-methoxy-3,6,9-trioxaundecyl, 11-ethoxy-3,6,9-trioxaundecyl, 15-ethoxy-4,8,12-trioxapentadecyl;
di(C₁-C₁₀-alkoxy-C₁-C₁₈-alkyl) such as diethoxymethyl or diethoxyethyl, C₁-C₆-alkoxycarbonyl-C₁-C₁₈-alkyl such as 2-(methoxycarbonyl)ethyl, 2-(ethoxycarbonyl)ethyl, 2-(n-butoxycarbonyl)ethyl,
di(C₁-C₆-alkoxycarbonyl)-C₁-C₁₈-alkyl such as 1,2-di(methoxycarbonyl)ethyl, hydroxy-C₁-C₁₈-alkyl such as 2-hydroxyethyl, 2-hydroxypropyl, 3-hydroxypropyl, 4-hydroxybutyl, 6-hydroxyhexyl, 2-hydroxy-2,2-dimethylethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl;
C₁-C₁₂-alkylsulfanyl-C₁-C₁₈-alkyl such as butylthiomethyl, 2-dodecylthioethyl, C₅-C₁₂-cycloalkyl-C₁-C₁₈-alkyl such as cyclopentylmethyl, 2-cyclopentylethyl, 3-cyclopentylpropyl, cyclohexylmethyl, 2-cyclohexylethyl, 3-cyclohexylpropyl, phenyl-C₁-C₁₈-alkyl, where the phenyl part of phenyl-C₁-C₁₈-alkyl is unsubstituted or substituted by one, two, three or four substituents selected independently from among C₁-C₆-alkyl, halogen, C₁-C₆-alkoxy and nitro, e.g. benzyl (phenylmethyl), 1-phenylethyl, 2-phenylethyl, 3-phenylpropyl, p-tolylmethyl, 1-(p-butylphenyl)ethyl, p-chlorobenzyl, 2,4-dichlorobenzyl, p-methoxybenzyl, m-ethoxybenzyl, phenyl-C(CH₃)₂₋, 2,6-dimethyl-phenylmethyl,
diphenyl-C₁-C₁₈-alkyl such as diphenylmethyl (benzhydryl);
triphenyl-C₁-C₁₈-alkyl such as triphenylmethyl;
phenoxy-C₁-C₁₈-alkyl such as 2-phenoxyethyl, 2-phenoxypropyl, 3-phenoxypropyl, 4-phenoxybutyl, 6-phenoxyhexyl; and
phenylthio-C₁-C₁₈-alkyl such as 2-phenylthioethyl.

When R¹ to R⁹ in the compounds of the formulae IV.a to IV.v are C₂-C₁₈-alkenyl, these are preferably selected from among C₂-C₆-alkenyl such as vinyl, 2-propenyl, 3-butenyl, cis-2-butenyl, trans-2-butenyl and C₂-C₁₈-alkenyl which is partially or fully substituted by fluorine.

When R¹ to R⁹ in the compounds of the formulae IV.a to IV.v are C₆-C₁₀-aryl, then R¹ to R⁹ are preferably phenyl or naphthyl, where phenyl or naphthyl is unsubstituted or substituted by one, two, three or four substituents selected independently from among halogen, C₁-C₁₅-alkyl, C₁-C₆-alkoxy, C₁-C₆-alkylsulfanyl, C₁-C₆-alkoxy-C₁-C₆-alkyl, C₁-C₆-alkylcarbonyl, amino, C₁-C₆-alkylamino, di(C₁-C₆-dialkyl)amino and nitro, e.g. phenyl, methylphenyl (tolyl), dimethylphenyl (xylyl) such as 2,6-dimethylphenyl, trimethylphenyl such as 2,4,6-trimethylphenyl, ethylphenyl, diethylphenyl, isopropyl-phenyl, tert-butylphenyl, dodecylphenyl, chlorophenyl, dichlorophenyl, trichlorophenyl, fluorophenyl, difluorophenyl, trifluorophenyl, tetrafluorophenyl, pentafluorophenyl, 2,6-dichlorophenyl, 4-bromophenyl, methoxyphenyl, dimethoxyphenyl, ethoxyphenyl, hexyloxyphenyl, 2,6-dimethoxyphenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl, tert-butylthiophenyl, α-naphthyl, β-naphthyl, methylnaphthyl, isopropylnaphthyl, chloronaphthyl, ethoxynaphthyl or partially fluorinated phenyl or perfluorinated phenyl.

When R¹ to R⁹ in the compounds of the formulae IV.a to IV.v are C₅-C₁₂-cycloalkyl, then R¹ to R⁹ are preferably unsubstituted cycloalkyl such as cyclopentyl or cyclohexyl; C₅-C₁₂-cycloalkyl which is substituted by one or two substituents selected independently from among C₁-C₆-alkyl, C₁-C₆-alkoxy, C₁-C₆-alkylsulfanyl or chlorine, e.g. butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl; C₅-C₁₂-cycloalkyl which is entirely or fully fluorinated.

When R¹ to R⁹ in the compounds of the formulae IV.a to IV.v are polycyclyl, then R¹ to R⁹ are preferably C₅-C₁₂-bicycloalkyl such as norbornyl or C₅-C₁₂-bicycloalkenyl such as norbornenyl.

When R¹ to R⁹ in the compounds of the formulae IV.a to IV.v are C₅-C₁₂-cycloalkenyl, then R¹ to R⁹ are preferably unsubstituted cycloalkenyl such as cyclopent-2-en-1-yl, cyclopent-3-en-1-yl, cyclohex-2-en-1-yl, cyclohex-1-en-1-yl, cyclohexa-2,5-dien-1-yl or partially or fully fluorinated cycloalkenyl.

When R¹ to R⁹ in the compounds of the formulae IV.a to IV.v are heterocycloalkyl having 5 or 6 ring atoms, then R¹ to R⁹ are preferably 1,3-dioxolan-2-yl, 1,3-dioxan-2-yl, 2-methyl-1,3-dioxolan-2-yl, 4-methyl-1,3-dioxolan-2-yl.

When R¹ to R⁹ in the compounds of the formulae IV.a to IV.v are heteroaryl, then R¹ to R⁹ are preferably furyl, thienyl, pyrryl, pyridyl, indolyl, benzoxazolyl, benzimidazolyl, benzothiazolyl. If it is substituted, hetaryl bears 1, 2 or 3 substituents selected independently from among C₁-C₆-alkyl, C₁-C₆-alkoxy and halogen, for example dimethylpyridyl, methylquinolyl, dimethylpyrryl, methoxyfuryl, dimethoxypyridyl or difluoropyridyl.

Particular preference is given to the radicals R¹ to R⁹ in the compounds of the formulae IV.a to IV.v each being, independently of one another, hydrogen, unbranched or branched C₁-C₁₈-alkyl which is unsubstituted or substituted by one or more hydroxy, halogen, phenyl, cyano, C₁-C₆-alkoxycarbonyl and/or sulfo groups, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, 2-hydroxyethyl, benzyl, 3-phenyl-propyl, 2-cyanoethyl, methoxycarbonylmethyl, ethoxycarbonylmethyl, n-butoxy-carbonylmethyl, tert-butoxycarbonylmethyl, 2-(methoxycarbonyl)ethyl, 2-(ethoxy-carbonyl)ethyl, 2-(n-butoxycarbonyl)ethyl, trifluoromethyl, difluoromethyl, fluoromethyl, pentafluoroethyl, heptafluoropropyl, heptafluoroisopropyl, nonafluorobutyl, nonafluoro-isobutyl, undecylfluoropentyl, undecylfluoroisopentyl or 6-hydroxyhexyl and 3-sulfo-propyl,

hydroxyethyloxyalkyl, radicals of oligoalkylene glycols and polyalkylene glycols, e.g. polyethylene glycols and polypropylene glycols and oligomers thereof having from 2 to 100 units and an H or C₁-C₈-alkyl as end group, for example R^{A}O—(CHR^{B}—CH₂—O)ₙ₋CHR^{B}—CH₂₋ or R^{A}O—(CH₂CH₂CH₂CH₂O)ₙ₋CH₂CH₂CH₂CH₂O—, where R^{A} and R^{B} are preferably H, methyl or ethyl and n is preferably from 0 to 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl; C₂-C₄-alkenyl such as vinyl and allyl; and
N,N-di-C₁-C₆-alkylamino such as N,N-dimethylamino and N,N-diethylamino.

Very particular preference is given to the radicals R¹ to R⁹ each being, independently of one another, hydrogen; C₁-C₁₈-alkyl such as methyl, ethyl, 1-butyl, 1-pentyl, 1-hexyl, 1-heptyl, 1-octyl; phenyl; 2-hydroxyethyl; 2-cyanoethyl; 2-(alkoxycarbonyl)ethyl such as 2-(methoxycarbonyl)ethyl, 2-(ethoxycarbonyl)ethyl or 2-(n-butoxycarbonyl)ethyl; N,N—(C₁-C₄-dialkyl)amino such as N,N-dimethylamino or N,N-diethylamino; chlorine or radicals of oligoalkylene glycol, e.g CH₃O—(CH₂CH₂O)ₙ—CH₂CH₂— or CH₃CH₂O—(CH₂CH₂O)ₙ₋CH₂CH₂₋, where n is from 0 to 3.

Preferred pyridinium ions are compounds of the formula IV.a in which one of the radicals R¹ to R⁵ is methyl, ethyl or chlorine and the remaining radicals R¹ to R⁵ are each H.

Further preferred pyridinium ions are compounds of the formula IV.a in which R³ is dimethylamino and the remaining radicals R¹, R², R⁴ and R⁵ are each H.

Further preferred pyridinium ions are compounds of the formula IV.a in which the radicals R¹ to R⁵ are each H.

Further preferred pyridinium ions are compounds of the formula IV.a in which R² is carboxy or carboxamide and the remaining radicals R¹, R², R⁴ and R⁵ are each H.

Further preferred pyridinium ions are compounds of the formula IV.a in which R¹ and R² or R² and R³ are together 1,4-buta-1,3-dienylene and the remaining radicals R¹, R², R⁴ and R⁵ are each H.

Particularly preferred pyridinium ions are pyridinium, 2-methylpyridinium, 2-ethylpyridinium, 5-ethyl-2-methylpyridinium and 2-methyl-3-ethylpyridinium and also 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)-pyridinium, 1-(1-tetradecyl)pyridinium, 1-(1-hexadecyl)pyridinium, 1,2-dimethyl-pyridinium, 1-ethyl-2-methylpyridinium, 1-(1-butyl)-2-methylpyridinium, 1-(1-hexyl)-2-methylpyridinium, 1-(1-octyl)-2-methylpyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethylpyridinium, 1-(1-dodecyl)-2-ethylpyridinium, 9-(1-tetradecyl)-2-ethylpyridinium, 1-(1-hexadecyl)-2-ethylpyridinium, 1,2-dimethyl-5-ethylpyridinium, 1,5-diethyl-2-methylpyridinium, 1-(1-butyl)-2-methyl-3-ethylpyridinium, 1-(1-hexyl)-2-methyl-3-ethylpyridinium and 1-(1-octyl)-2-methyl-3-ethyl-pyridinium, 1-(1-dodecyl)-2-methyl-3-ethylpyridinium, 1-(1-tetradecyl)-2-methyl-3-ethyl-pyridinium and 1-(1-hexadecyl)-2-methyl-3-ethylpyridinium.

Preferred pyridazinium ions are compounds of the formula IV.b in which the radicals R¹ to R⁴ are each H or in which one of the radicals R¹ to R⁴ is methyl or ethyl and the remaining radicals R¹ to R⁴ are each H.

Preferred pyrimidinium ions are compounds of the formula IV.c in which R¹ is H, methyl or ethyl and R² to R⁴ are each, independently of one another, H or methyl or in which R¹ is H, methyl or ethyl, R² and R⁴ are each methyl and R³ is H.

Preferred pyrazinium ions are compounds of the formula IV.d in which R¹ is H, methyl or ethyl and R² to R⁴ are each, independently of one another, H or methyl or in which R¹ is H, methyl or ethyl and R² and R⁴ are each methyl and R³ is H or in which R¹ to R⁴ are each methyl or in which R¹ to R⁴ are each H.

Preferred imidazolium ions are compounds of the formula IV.e in which R¹ is H, methyl, ethyl, 1-propyl, 1-butyl, 1-pentyl, 1-hexyl, 1-octyl, 2-hydroxyethyl or 2-cyanoethyl and R² to R⁴ are each, independently of one another, H, methyl or ethyl.

Particularly preferred imidazolium ions of the formula IV.e are 1-methylimidazolium, 1-ethylimidazolium, 1-(1-propyl)imidazolium, 1-(1-allyl)imidazolium, 1-(1-butyl)-imidazolium, 1-(1-octyl)imidazolium, 1-(1-dodecyl)imidazolium, 1-(1-tetradecyl)-imidazolium, 1-(1-hexadecyl)imidazolium, 1,3-dimethylimidazolium, 1,3-diethyl-imidazolium, 1-ethyl-3-methylimidazolium, 1-(1-butyl)-3-methylimidazolium, 1-(1-butyl)-3-ethylimidazolium, 1-(1-hexyl)-3-methylimidazolium, 1-(1-hexyl)-3-ethylimidazolium, 1-(1-hexyl)-3-butylimidazolium, 1-(1-octyl)-3-methylimidazolium, 1-(1-octyl)-3-ethyl-imidazolium, 1-(1-octyl)-3-butylimidazolium, 1-(1-dodecyl)-3-methylimidazolium, 1-(1-dodecyl)-3-ethylimidazolium, 1-(1-dodecyl)-3-butylimidazolium, 1-(1-dodecyl)-3-octylimidazolium, 1-(1-tetradecyl)-3-methylimidazolium, 1-(1-tetradecyl)-3-ethylimidazolium, 1-(1-tetradecyl)-3-butylimidazolium, 1-(1-tetradecyl)-3-octylimidazolium, 1-(1-hexadecyl)-3-methylimidazolium, 1-(1-hexadecyl)-3-ethylimidazolium, 1-(1-hexadecyl)-3-butylimidazolium, 1-(1-hexadecyl)-3-octylimidazolium, 1,2-dimethylimidazolium, 1,2,3-trimethyl-imidazolium, 1-ethyl-2,3-dimethylimidazolium, 1-(1-butyl)-2,3-dimethylimidazolium, 1-(1-hexyl)-2,3-dimethylimidazolium, 1-(1-octyl)-2,3-dimethylimidazolium, 1,4-dimethyl-imidazolium, 1,3,4-trimethylimidazolium, 1,4-dimethyl-3-ethylimidazolium, 3-methyl-imidazolium, 3-ethylimidazolium, 3-n-propylimidazolium, 3-n-butylimidazolium, 1,4-dimethyl-3-octylimidazolium, 1,4,5-trimethylimidazolium, 1,3,4,5-tetramethyl-imidazolium, 1,4,5-trimethyl-3-ethylimidazolium, 1,4,5-trimethyl-3-butylimidazolium, 1,4,5-trimethyl-3-octylimidazolium, 1-prop-1-en-3-yl-3-methylimidazolium and 1-prop-1-en-3-yl-3-butylimidazolium.

Preferred pyrazolium ions are compounds of the formulae IV.f, IV.g and IV.g′ in which R¹ is H, methyl or ethyl and R² to R⁴ are each, independently of one another, H or methyl.

Further preferred pyrazolium ions are compounds of the formula IV.h in which R¹ to R⁴ are each, independently of one another, H or methyl.

Particularly preferred pyrazolium ions are 1,4-dimethylpyrazolium and 1,2,4-trimethyl-pyrazolium.

Preferred 1-pyrazolinium ions are compounds of the formula IV.i in which R¹ to R⁶ are each, independently of one another, H or methyl.

Preferred 2-pyrazolinium ions are compounds of the formulae IV.j and IV.j′ in which R¹ is H, methyl, ethyl or phenyl and R² to R⁶ are each, independently of one another, H or methyl.

Preferred 3-pyrazolinium ions are compounds of the formulae IV.k. and IV.k′ in which R¹ and R² are each, independently of one another, H, methyl, ethyl or phenyl and R³ to R⁶ are each, independently of one another, H or methyl.

Preferred imidazolinium ions are compounds of the formula (IV.I ) in which R¹ and R² are each, independently of one another, H, methyl, ethyl, 1-butyl or phenyl, R³ and R⁴ are each, independently of one another, H, methyl or ethyl and R⁵ and R⁶ are each, independently of one another, H or methyl.

Further preferred imidazolinium ions are compounds of the formulae IV.m and IV.m′ in which R¹ and R² are each, independently of one another, H, methyl or ethyl and R³ to R⁶ are each, independently of one another, H or methyl.

Further preferred imidazolinium ions are compounds of the formulae IV.n and IV.n′ in which R¹ to R³ are each, independently of one another, H, methyl or ethyl and R⁴ to R⁶ are each, independently of one another, H or methyl.

Preferred thiazolium ions are compounds of the formulae IV.o and IV.o′ in which R¹ is H, methyl, ethyl or phenyl and R² and R³ are each, independently of one another, H or methyl.

Preferred oxazolium ions are compounds of the formula IV.p in which R¹ is H, methyl, ethyl or phenyl and R² and R³ are each, independently of one another, H or methyl.

Preferred 1,2,4-triazolium ions are compounds of the formulae IV.q, IV.q′ and IV.q″ in which R¹ and R² are each, independently of one another, H, methyl, ethyl or phenyl and R³ is H, methyl or phenyl.

Preferred 1,2,3-triazolium ions are compounds of the formulae IV.r, IV.r′ and IV.r″ in which R¹ is H, methyl or ethyl, R² and R³ are each, independently of one another, H or methyl or R² and R³ are together 1,4-buta-1,3-dienylene.

Preferred pyrrolidinium ions are compounds of the formula IV.s in which R¹ is H, methyl, ethyl or phenyl and R² to R⁹ are each, independently of one another, H or methyl.

Preferred imidazolidinium ions are compounds of the formula IV.t in which R¹ and R⁴ are each, independently of one another, H, methyl, ethyl or phenyl and R², R³ and R⁵ to R⁸ are each, independently of one another, H or methyl.

Preferred diazabicycloalkenium ions of the formulae IV.u and IV.v are selected from among cationic derivatives of 1,5-diazabicyclo[4.3.0]non-5-ene (DBN) and 1,8-diaza-bicyclo[5.4.0]undec-7-ene (DBU).

In a particularly preferred embodiment of the process of the invention, an ionic liquid IL having at least one cation selected from among the abovementioned imidazolium ions and the abovementioned pyrazolium ions is worked up. The cation of the ionic liquid is very particularly preferably selected from among the abovementioned imidazolium ions. As regards preferred imidazolium ions and pyrazolium ions, reference may be made to what has been said above in its full scope.

The present invention is illustrated below with the aid of nonlimiting examples.

### EXAMPLES

To determine the pH values of ionic liquids reported below, 1 ml of the ionic liquid was in each case diluted with water to a total volume of 10 ml and the pH of the mixture obtained in this way was determined by means of a glass electrode.

### Example 1

### Work-Up of Acidic EMIM Tosylate

a) Neutralization of EMIM tosylate

1-Ethyl-3-methylimidazolium tosylate (EMIM tosylate) was prepared by reaction of EMIM chloride with p-toluenesulfonic acid. The EMIM tosylate obtained in this way had a pH of 2.5 (determined by the above method). A solution of EMIM butoxide (1 M in methanol; 5.186 g) was added to this EMIM tosylate (pH=2.5; 50.3 g). The mixture obtained had a pH of 6.9 (determined by the above method). Methanol and the butanol formed in the neutralization were removed under reduced pressure at 120° C. on a rotary evaporator. The residue had a pH of 6.7 (determined by the above method).

b) Testing of the properties of EMIM tosylate (pH=6.9)

A mixture of trimethyl borate (0.642 g), methanol (5.774 g) and EMIM tosylate from example 1.a) (pH=6.9; 57.739 g) was placed in a stainless steel crucible (1.457 I) and subjected to an adiabatic pressure measurement. The adiabatic pressure test is an adiabatic runaway test in a reaction vessel with further introduction of pressure and heat. An initial pressure of 2 bar (absolute) was set by means of nitrogen. The internal temperature of the stainless steel crucible was maintained at 130° C. for 24 hours and at 170° C. for a further 59 hours. No pressure increase took place during this time. No dimethyl ether could be detected as decomposition product by headspace GC-MS analysis. The mixture comprising the worked-up ionic liquid is thermally stable.

3. Testing of the properties of EMIM tosylate (pH=2.5) (comparative example)

A mixture of trimethyl borate (0.327 g), methanol (2.941 g) and EMIM tosylate (pH=2.5; 29.412 g) was placed in a stainless steel crucible (1.457 I) and subjected to an adiabatic pressure measurement. An initial pressure of 2 bar (absolute) was set by means of nitrogen. The stainless steel crucible was heated to an internal temperature of 130° C. The pressure rose to about 10 bar within the first hour and to over 50 bar within 40 hours. Dimethyl ether could be detected as decomposition product by headspace GC-MS analysis.

1. A process for the work-up of an ionic liquid comprising a compound represented by formula (I),
[A]⁺(1/n)*[Y]⁹⁻ (I)
where [A]⁺is a quaternary ammonium cation and (1/n)* [Y]ⁿ⁻ is one anion equivalent of an n-fold charged anion,
where the ionic liquid has an acidic or basic pH,
which process comprises adding a conjugate acid-base pair comprising a compound represented by formula (II),
[A]⁺[X]⁻ (II)
where [A]⁺has the meaning given for the ionic liquid and [X]⁻ is a conjugate base, to the ionic liquid.

2. The process according to claim 1, wherein the ionic liquid has a pH in the range from 1 to 6.9 and the conjugate acid-base pair of the formula (II) is a basic compound.

3. The process according to claim 2, wherein the conjugate base [X]⁻ is an anion of the formula ⁻OR¹, where R¹ is alkyl or cycloalkyl.

4. The process according to claim 3, wherein R¹ is C₁-C₄-alkyl.

5. The process according to claim 1, wherein the neutralization product of the formula X-H is separated off from the ionic liquid.

6. The process according to claim 5, wherein the neutralization product of the formula X-H is separated off by distillation.

7. The process according to claim 2, wherein [Y]ⁿ⁻ in the compound represented by formula (I) is selected from the group consisting of a compound of formulae (R^{a})SO₃⁻, (R^{a})SO₂⁻, (R^{a}) PO₃²⁻, (R^{a})(R^{b})PO₂⁻, (R^{a})PO₂²⁻,(R^{a})(R^{b})PO⁻ and (R^{a})PO²⁻,
where R^{a} and R^{b} are each, independently of one another, OH, alkyl, alkoxy, aryl, aryloxy, cycloalkyl, cycloalkoxy, heterocycloalkyl, heterocycloalkyloxy, heteroaryl or heteroaryloxy.

8. The process according to claim 7, wherein [Y]ⁿ⁻is a compound of the formulae (R^{a})SO₃⁻ or (R^{a})(R^{b})PO₂⁻, where R^{a} and R^{b} are each, independently of one another, alkyl, alkoxy, aryl or aryloxy.

9. The process according to claim 8, wherein [Y]ⁿ⁻is a compound of the formula (R^{a})SO₃⁻.

10. The process according to claim 1, wherein [A]⁺ in the compounds of the formulae (I) and (II) is a compound having a molar mass of less than 1000 g/mol.

11. The process according to claim 1, wherein [A]⁺is an optionally substituted heterocyclic cation.

12. The process according to claim 11, wherein [A]⁺is selected from the group consisting of optionally substituted pyridinium, pyridazinium, pyrimidinium, pyrazinium, imidazolium, pyrazolium, thiazolium, oxazolium, 1,2,4-triazolium, 1,2,3-triazolium, pyrrolidinium, imidazolidinium and diazabicycloalkenium ions.

13. The process according to claim 12, wherein [A]⁺is an optionally substituted imidazolium or pyrazolium ion.

14. The process according to claim 13, wherein [A]⁺is an optionally substituted imidazolium ion.

15. The process according to claim 1, further comprising separating a neutralization product of the formula X-H is separated off from the ionic liquid.

16. The process according to claim 5, wherein said separating of the neutralization product of the formula X-H is carried out by distillation.
